(19) United States
(12) Patent Application Publication
(10) Pub. No.: US 2020/0013507 A1
(43) Pub. Date: Jan. 9, 2020

(54) BEST FIT CONTENT DELIVERY IN CARE PLAN ENVIRONMENT

(71) Applicant: Preventice Technologies, Inc, Rochester, MN (US)

(72) Inventors: Rudiger Da-Ming BRAUN, Rochester, MN (US); Richard M. SMITH, Oronoco, MN (US)

(21) Appl. No.: 16/030,636

(22) Filed: Jul. 9, 2018

(51) Int. Cl. G16H 50/20; G06F 17/30; G06F 7/02

(52) U.S. Cl. CPC G16H 50/20; G06F 7/026; G06F 17/30309

(57) ABSTRACT

Embodiments disclosed herein are directed to methods of administering information in a care plan to a patient. Embodiments include receiving a request specifying an identifier for a digital content item to be delivered as part of administering an individualized care plan for a patient. The processor matches the document identifier to a catalogue in a digital content library. The catalogue includes a plurality of versions of the digital content item. The processor selects one of the versions of the digital content item in the catalogue based on metadata for the patient. The patient metadata includes personal information about the patient. The processor transmits the selected digital content item to a device of the patient.

### BACKGROUND

### Field

Embodiments presented herein generally describe techniques related to health care, and more specifically, for administering best fit content in the individualized care plan for a patient.

### Description of the Related Art

In the health care field, a care plan is a set of tasks provided by a health care practitioner (e.g., a doctor) to a patient. Historically, care plans are a written document that provides directions and routines for a patient to follow to manage certain health conditions. The care plan may include a set of tasks (e.g., exercise for a given duration) for the patient to perform, content that educates the patient about a diagnosed condition (e.g., brochures describing the diagnosed condition), and logs for the patient to periodically record information in (e.g., weight, blood pressure, etc.). As an example, a doctor might create a care plan for a patient with hypertension that includes several brochures describing hypertension and hypertension treatment and assigned tasks such as walking on a treadmill for thirty minutes each morning, drinking a glass of water every three hours, and recording blood pressure at the end of each day. Thus, as part of the treatment of the condition, the patient is expected to adhere to the tasks listed in the care plan and to then follow up with the doctor in a subsequent appointment to assess the patient's progress and adherence to the care plan, and to make any needed adjustments to the care plan accordingly.

Current care platforms are limited in providing the same version of the desired content to users having different user metadata. For example, given a care plan that includes a portable document format (PDF) on high-level information about diabetes, a first user having a grade-school education may have more difficulty than a second user having a post-doctoral education understanding the high-level information in the PDF. Using the same example, a first user having a vision problem have more difficulty (or be unable entirely) to read the high-level information compared to a second user without any vision problems. Thus, the current landscape provides a one-size-fits-all approach in providing the same information to users having differing user metadata.

### SUMMARY

Embodiments disclosed herein are directed to methods of administering information in a care plan to a patient. Embodiments include receiving a request specifying an identifier for a digital content item to be delivered as part of administering an individualized care plan for a patient. The processor matches the document identifier to a catalogue in a digital content library. The catalogue includes a plurality of versions of the digital content item. The processor selects one of the versions of the digital content item in the catalogue based on metadata for the patient. The patient metadata includes personal information about the patient. The processor transmits the selected digital content item to a device of the patient.

Additional embodiments provide a computer-readable medium and system for carrying out the aforementioned method.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example computing environment, according to one embodiment.

FIG. 2 further illustrates the care platform server described in FIG. 1, according to one embodiment.

FIG. 3 illustrates a method for delivering best fit content, according to one embodiment.

FIG. 4 illustrates a method for delivering best fit content, according to one embodiment.

FIG. 5 a flow diagram of a block of the method of FIG. 3 for choosing a digital content item from the catalogue based on the metadata, according to one embodiment.

FIG. 6 illustrates a method for delivering best fit content, according to one embodiment.

FIG. 7 illustrates a method for delivering best fit content, according to one embodiment.

FIG. 8 illustrates a care platform server configured to administer best fit content in the individualized care plan for a patient, according to one embodiment.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Current approaches for providing a care plan for a patient to follow and monitoring the patient's adherence are rigid. For example, in many cases, a physician who has diagnosed a patient with a particular condition may provide the patient with a generalized care plan commonly used to address that condition, e.g., pre-printed documents that are not tailored to any particular patient but, rather, generally address the diagnose medical condition. As a result, such printed care plans do not allow the physician to customize the care plan beyond annotating the care plan along the physical margins.

Further, to address multiple conditions, a physician typically has to print separate care protocol pamphlets addressing each of multiple medical conditions the patient has been diagnosed with. Manually annotating each of the pre-printed documents is particularly inconvenient when the physician must reconcile multiple care protocols for multiple different medical conditions the patient has been diagnosed with, where treatment recommendations within one of the protocols may differ from or even conflict with the treatment recommendation for another of the care protocols. Additionally, some tasks in each of the printed pamphlets may overlap (e.g., two different care plans may instruct a patient to take a certain dosage of aspirin at a given time of day), a patient may have difficulty understanding the tasks to perform, leading to poor compliance. Moreover, in some situations, the tasks for treating the various diagnosed conditions may conflict with one another, leaving the patient unsure as to how to reconcile the conflict.

A care platform allows a physician to design a care plan tailored specifically for an individual patient. The care plan may include multiple care plan protocols, each of which addressing a respective medical condition the patient has been diagnosed with. Generally, a care plan protocol describes the treatment of a particular medical condition. For instance, a care plan could specify a set of specific tasks for a patient to follow to manage the particular medical condition and could divide the tasks by phases and schedules. Generally, different care plan protocols may be available for each of a variety of conditions, such as congestive heart failure, diabetes, sprained ankle, etc.

A physician may assign multiple care plan protocols to a patient suffering from multiple medical conditions. To do so, the physician may configure templates for care plan protocols corresponding to the patient's conditions. Each care protocol template can further provide a general set of tasks to follow for a given condition. The physician may customize each template specifically for the patient. For example, assume a care protocol template for congestive heart failure recover specifies a number of tasks to be performed at a specified interval (e.g., daily), such as walk for fifteen minutes, take prescribed medicine, record blood pressure, and record weight. If the physician deems that the patient is already within a healthy weight, the physician may remove the “record weight” task from the protocol. Further the physician may also adjust the length of the time patient should walk. The physician may additionally insert extra tasks to the protocol.

The care platform may consolidate the configured care plan protocols into one overall care plan for the patient. However, it is possible that some tasks and phases of different care plan protocols overlap. As such, the care platform may perform operations to reconcile any conflicts between care plan protocols as part of the care plan creation. For example, a care plan protocol for high blood pressure and another for diabetes may include a step for walking for ten minutes a day. Both protocols may also include a step for taking two aspirin pills in the morning, and including two separate steps for taking aspirin may yield unintended consequences related to a dosage that the patient should be taking. As such, the care platform could consolidate the two separate tasks into a single task of taking two aspirin pills in the morning.

Once created, the care platform may determine a set of monitoring devices to use in collecting data for the observation metrics specified within the care plan. Generally, the care platform can select at least one device for every observation metric. The patient (or care provider) can select any suitable device(s) for collecting data for the specified observation metrics.

In one embodiment, a mobile device (e.g., a smart phone or tablet device) is used to facilitate communication between the monitoring devices and the care platform server. For example, an application(s) deployed on a mobile device could communicate with each of the monitoring devices (e.g., wirelessly using Bluetooth® communications) to collect monitored data from each of the devices. The application on the mobile device could then transmit the collected data to the care platform server over a communications network (e.g., the Internet). Doing so allows the collected data to be transmitted to the care platform server without requiring each monitoring device to have a separate connection to the communications network, and thus allows monitoring devices not capable of connecting to the communications network to still be used.

Once the monitoring devices are determined, the care platform can transmit the care plan to the application deployed on the mobile device. Through the device, the patient may access the care plan and understand the tasks to perform. Moreover, information can be provided to the patient through the mobile device as part of the administration of the care plan. For example, such information can be provided via a display device of the mobile device, through one or more speaker devices of the mobile device, other input/output devices on the mobile device, or some combination therefor. The application can record the information to the care plan and relay that information to the care platform. As a result, the physician can monitor the patient's adherence to the care plan.

The delivery of the content efficiently to the application is a high priority for the care platform. However, it is possible that the version of the content provided to the application is not optimized for a given user. For example, the content may be pushed to applications on varying device types, to users of differing languages, disabilities, sex, age, location, medical history, and similar personal information. The personal information can be considered “metadata” for the respective user. Current care platforms are limited in providing the same version of the desired content to users having different user metadata. For example, given a care plan that includes a portable document format (PDF) on high-level information about diabetes, a first user having a grade-school education may have more difficulty than a second user having a post-doctoral education understanding the high-level information in the PDF. Using the same example, a first user having a vision problem have more difficulty (or be unable entirely) to read the high-level information compared to a second user without any vision problems. Thus, the current landscape provides a one-size-fits-all approach in providing the same information to users having differing user metadata.

Embodiments presented herein describe techniques for administering a customized health care plan for an individual. In one embodiment, the care platform includes one or more care plan protocols addressing a respective medical condition. The care platform may transmit one or more documents to a user with information about the respective medical condition. The care platform is able to transmit a “best fit” document to the user based on the metadata of the user requesting the information. Generally, the metadata may include a variety of variables such as medical history, institution history, sex, age, location, education history, etc. The best fit enables a client to receive and view information specifically tailored to the client. Doing so will provide a more efficient experience for the overall care platform because the user will receive only items fitting a best-fit criteria.

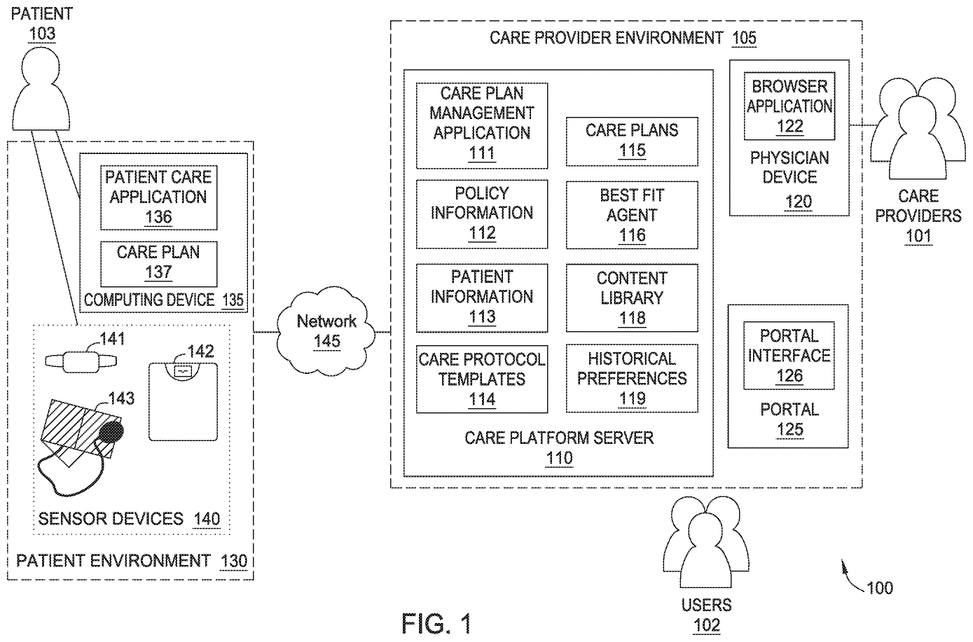

FIG. 1 illustrates an example computing environment 100, according to one embodiment. As shown, the computing environment 100 may include a care provider environment 105 and a patient environment 130, each connected to one another via a network 145. The environments 105 and 130 allow a patient 103 to communicate with a care provider 101 (e.g., a physician).

The care provider environment 105 includes a care platform server 110, a physician device 120, and a portal 125. Each of the care platform server 110, physician device 120, and portal 125 may be a physical computing system or may be a virtual computer instance (e.g., executing in a cloud computing platform). A care provider may use the physician device 120 to access (e.g., via a browser application 122) a portal user interface 126 hosted by the portal 125. The portal user interface 126 itself provides users 102 (e.g., the care providers 101, the patient, authorized members of the patient's family, etc.) with access to the care platform server 110.

The care platform server 110 includes various applications and data that allow a care provider 101 to create and manage a care plan for a patient 103. As shown, the care platform server 110 includes a care plan management application 111, policy information 112, patient metadata 113, care protocol templates 114, care plans 115, best fit agent 116, content library 118, and historical preferences 119. The care plan management application 111 generates care plans 115 based on care protocol templates 114.

A care plan 115 may be created based on one or more care protocols, with each of the care protocols relating to a respective medical condition the patient has been diagnosed with. A care protocol is a set of tasks that a patient 103 follows to manage a certain condition, metrics that the care plan management application 111 monitors, objectives for the patient to meet, and the like. For instance, a care protocol may target recover from a heart attack. Another care protocol may treat diabetes. Tasks associated with a care protocol may include steps such as exercising for a specified duration or taking medication at a certain time of day.

To generate a care plan, a care provider 101 may configure care protocol templates 114 corresponding to medical conditions the patient 103 are diagnosed with. To do so, the care provider 101 (e.g., via the portal user interface 126) selects one or more care protocol templates 114 to associate with the patient 103. The care plan management application 111 populates a care plan with tasks, triggers, and monitoring thresholds as specified by the selected care protocol templates 114. The portal user interface may display the selected care protocol templates 114, where the care provider 101 may customize various facets of each selected template 114, such as tasks and thresholds. For example, the care provider 101 may customize a task instructing a patient to check blood pressure every morning. The care provider 101 may adjust the task so that the patient checks blood pressure twice a day. In addition, the care provider 101 may adjust thresholds associated with that task, such that the care platform server 110 alerts the care provider 101 if a threshold blood pressure is reached.

The care plan management application 111 generates a care plan 115 for a patient 103 based on the customizations made by the care provider 101. Once generated, the care plan management application 111 may store the care plan 115 on the care platform server 110. Further, the care plan management application 111 transmits the care plan 115 to a computing device 135 (e.g., to a patient care application 136 executing on the computing device 135) of the patient 103. For example, the computing device 135 may be a mobile device, a tablet device, a personal computer, a television, or the like.

The care plan 115 may include one or more documents that relate to the user's ailments and/or treatments for their ailments. For example, the one or more documents may include general information about the user's ailments (e.g., information about diabetes) or information for the treatment of their ailments (e.g., a diet plan for diabetes). Each of the one or more documents corresponds to one or more catalogues in the content library 118. The content library 118 stores one or more catalogues. Each catalogue includes one or more documents, wherein each document provides the same general information in varying formats. For example, the catalogue corresponding to “Generic Diabetes Information” may include the “Generic Diabetes Information” in PDF, PowerPoint, way, mp3, jpeg formats, as well as in various versions corresponding to different educational levels.

The best fit agent 116 is configured to determine which version of information in corresponding catalogue of the content library 118 is the digital content item to transmit for the user requesting a digital content item. The best fit agent 116 does this by looking at the patient metadata 113, and determining which version of information is most appropriate for the user. For example, the best fit agent 116 may determine that the version of “Generic Diabetes Information” that is an audio version may be the digital content item to transmit for a person with a sight deficiency. In another example, the best fit agent 116 may determine that the version of “Generic Diabetes Information” that is detailed with scientific information may be the digital content item to transmit for a person with a post-doctorate education. In another example, the best fit agent 116 may determine which digital content item to send to the patient based on the historical preferences 119. The historical preferences 119 store the patient's previous habits of consuming information. For example, historical preferences may include choosing a more sophisticated explanation of the information, choosing a text based digital content item as opposed to an audio digital content item, choosing a digital content item with large text as opposed to a digital content item with smaller text, and the like.

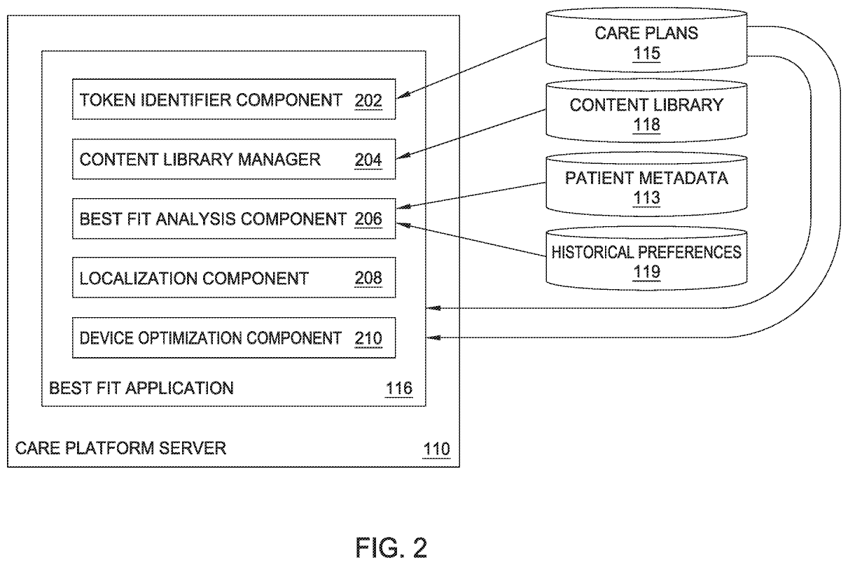

FIG. 2 further illustrates the care platform server 110, according to one embodiment. As stated, the best fit agent 116 can be configured to pick a digital content item for information in a care plan 115 based off patient metadata. As shown, the best fit agent 116 includes a document identifier component 202, a content library manager 204, best fit analysis component 206, a localization component 208, and device optimization component 210. When the best fit agent 116 receives a request for a digital content item from the user, the document identifier component 202 scans item requested for a document identifier. For example, when the user requests a digital content item for a document on “Generic Diabetes Information,” the document identifier component 202 scans the document for the identifier corresponding to “diabetes.” The document identifier corresponds to one or more catalogues in the content library 118. The content library manager 204 is configured to map the document identifier to a corresponding catalogue in the content library. For example, the content library manager 204 maps the document identifier “diabetes” to the diabetes catalogue in the content library 118.

The best fit analysis component 206 determines which version of the information in the catalogue is the digital content item to push to the user. In one embodiment, the best fit analysis component 206 communicates with the patient metadata 113 to determine the digital content item from the catalogue. In one embodiment, the best fit analysis component 206 communicates with the historical preferences 119 to determine the digital content item from the catalogue. For example, the best fit analysis component 206 considers patient metadata that may include age, sex, location, education level, institution history, medical history, and other data relating to personal information of the patient.

The localization component 208 and the device optimization component 210 are configured to provide the version of the digital content item to the user that best matches the patient as described by the patient metadata. The localization component 208 is configured to localize the content for the user. For example, the localization component 208 may determine the location of the user to determine the language in which the information in the digital content item should be translated. The device optimization component 210 is configured to determine the device specifications on which the user requested the best fit information. For example, the device optimization component 210 may be configured to determine the resolution of the screen of the requesting device. In another example, the device optimization component 210 may be configured to determine the streaming speed of the device.

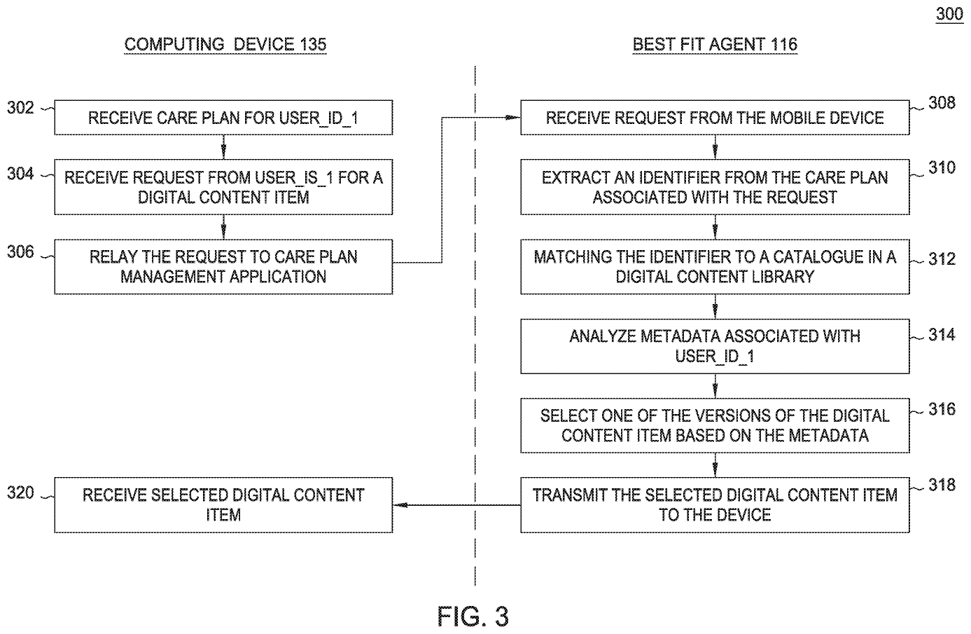

FIG. 3 illustrates a method 300 for delivering best fit content, according to one embodiment. As shown, the method 300 begins at block 302, where the computing device receives a care plan for User_ID_1. For example, such a care plan was generated by a health care provider using the care platform server 110. As discussed above, the care plan could specify assigned tasks for the patient to perform, a number of observational metrics to monitor, observational thresholds that, when met by the collected observational metric data, signify the occurrence of an event, and treatment plans specifying a treatment protocol for responding to the occurrence of such an event.

Upon receiving the care plan, the computing device may receive a digital content item request corresponding to an item in the care plan (block 304). For example, the request may be directed to general treatment information for diabetes. In another example, the request may be directed to information regarding physical therapy at-home exercises. The digital content item request may be responsive to user difficulties with the current format of the item in the care plan. For example, given an item on diabetes that may be rather terse and directed towards lower level users, the requesting user may want more detailed directives on his or her diabetes treatment. The computing device then relays the request to the best fit agent 116 (block 306).

The best fit agent 116 receives the request from the computing device (block 308). The best fit application extracts a document identifier from the care plan associated with the request (block 310). The document identifier corresponds to a given condition. For example, the document identifier may correspond to diabetes, heart disease, pancreatic cancer, torn Achilles tendon rehab, etc. The best fit application then matches the document identifier extracted in block 308 to a catalogue in the content library 118 (block 312). The content library 118 includes one or more catalogues, wherein each catalogue corresponds to a given condition. The catalogues contain equivalent forms of the same information in a variety of versions. For example, a diabetes catalogue may contain equivalent forms of an acceptable diet in PDF, PowerPoint, and mp3 form.

The best fit agent 116 analyzes metadata associated with the User_ID_01 to determine which version of the information requested is a digital content item (block 314). The best fit agent 116 looks at the patient metadata 113. For example, the patient metadata 113 that includes, but is not limited to, sex, age, education level, medical history, institutional history, medical state, and other factors associated with the user. The best fit agent 116 chooses a digital content item from the catalogue based on the analyzed metadata (block 316). For example, given metadata that includes a medical history of blindness, the best fit application will choose as a digital content item the version of information in audio format. In another example, given metadata that includes an educational history detailing a grade-school education level, the best fit application will choose as a digital content item the version of information that is of a “lower reading level.” The best fit agent 116 pushes the digital content item to the computing device (block 318). The computing device receives the digital content item from the best fit application (block 320).

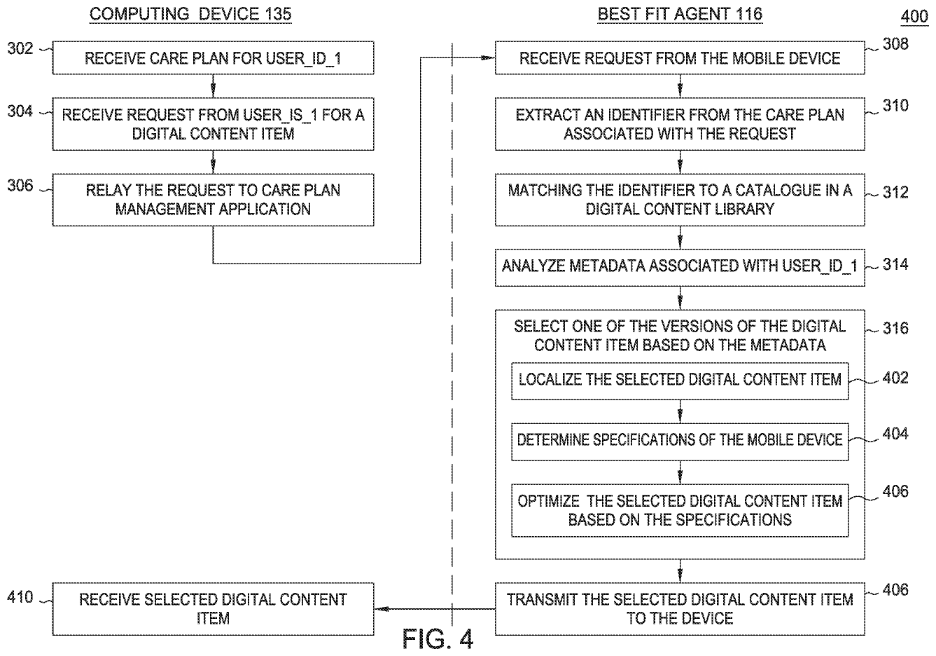

FIG. 4 illustrates a method 400 for delivering best fit content, according to one embodiment. The method 400 includes the blocks 302-316 from FIG. 3. In the embodiment shown in FIG. 4, block 316 includes sub-blocks 402-406. At block 402, the localization component 208 localizes the digital content item. For example, the localization component 208 may localize the digital content item based off the area code of User_ID_1. In another example, the localization component 208 may localize the digital content item based off the Internet Protocol (IP) address of the device.

The device optimization component 210 then determines the specifications of the computing device (block 404). For example, the device optimization component 210 may determine the screen size and load times of the patient's device, and adjust the best fit content item accordingly. Continuing with this example, the device optimization component may take into account the design of the best fit content item when pushing the best fit content item to the patient's device. In another example, the device optimization component 210 may determine the speed of the device, and provide an appropriate video to the patient's device that will not buffer and pause as often as a higher quality video.

The device optimization component 210 then chooses an optimized version of the digital content item based on the determined specifications of the computing device (block 406). For example, in response to determining that the computing device is a tablet having a high-screen resolution, the device optimization component 210 may choose a version that allows a user to choose a higher-resolution video. In another example, in response to determining that the computing device is a tablet having a high-screen resolution, the device optimization component 210 may choose a version that allows the user to choose between videos of varying resolution. The best fit agent 116 pushes the digital content item to the mobile device (block 408). The computing device receives the digital content item from the best fit application (block 410). In another embodiment, blocks 402-406 may be performed before blocks 308-314 in FIG. 3. In yet another embodiment, blocks 402-406 may be performed simultaneously, or in parallel to, blocks 308-314 in FIG. 3.

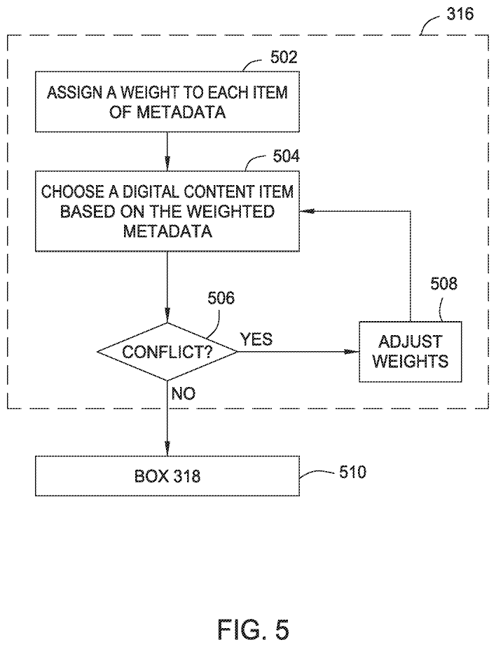

FIG. 5 a flow diagram of block 316 of method 300 for choosing the digital content item from the catalogue based on the metadata, according to one embodiment. Block 316 in method 300 includes sub-blocks 502-508. The best fit agent 116 may assign a weight to each item of metadata corresponding to the user. In one embodiment, the weights may be pre-set to differentiate among items of metadata. For example, the best fit agent 116 may weigh metadata according to personal disabilities greater than metadata according to age. Continuing with the example, the best fit agent 116 will put more importance in a user having a blindness disability compared to the user's age being in the mid-20s range. Weighing each item of metadata allows for a more precise digital content item.

The best fit agent 116 then chooses a digital content item based on the weighted metadata (block 504). For example, the best fit agent 116 may provide a point system to each item of metadata. Continuing with the example, the version of the information that has the highest score may then correspond to the digital content item. In another example, the best fit application may choose as the digital content item that version of the information that corresponds to the highest ranking metadata.

After the best fit agent 116 chooses the digital content item based on the weighted metadata, the best fit agent 116 determines whether there are any conflicts between the digital content item chosen and the user (block 506). For example, as discussed above, the best fit application may choose the digital content item based on a highest total score of a version of information. Continuing with the example, the best fit agent 116 may choose an audio version of the information for a user having a hearing deficiency because other items of metadata, as a whole, outweighed the hearing deficiency. If the best fit agent 116 determines that the conflict exists, then the best fit application may adjust the weights assigned to each item of metadata (block 508). Continuing with the above example, the best fit agent 116 may assign a higher weight to the hearing deficiency. If the best fit agent 116 determines that a conflict does not exist, then the best fit application continues to block 318 (block 510).

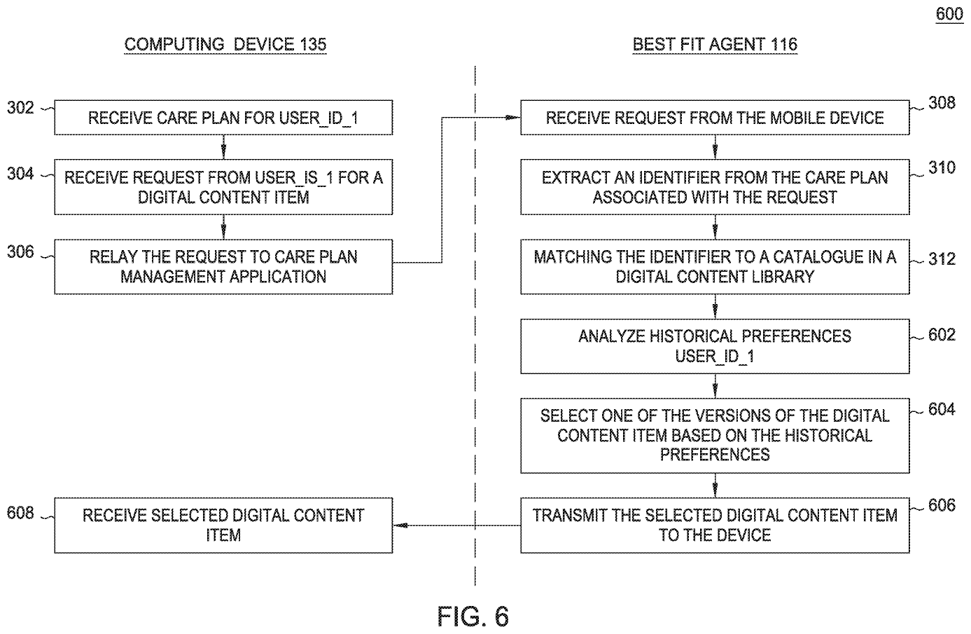

FIG. 6 illustrates a method 600 for delivering best fit content, according to one embodiment. The method 600 includes the blocks 302-312 from FIG. 3. After the best fit agent 116 matches the identifier to a catalogue in the library, the best fit agent 116 analyzes the historical preferences of the user (block 602). For example, the best fit agent 116 analyzes whether the user repeatedly selects a particular form of content (e.g., a high sophistication content, video as opposed to text, etc.). The best fit agent 116 is able to account for this preference, by selecting the specific form of content based off of the user's historical preferences (block 604). The best fit agent 116 then pushes the content item to the computing device (block 606). The computing device receives the digital content item from the best fit agent 116 (block 608).

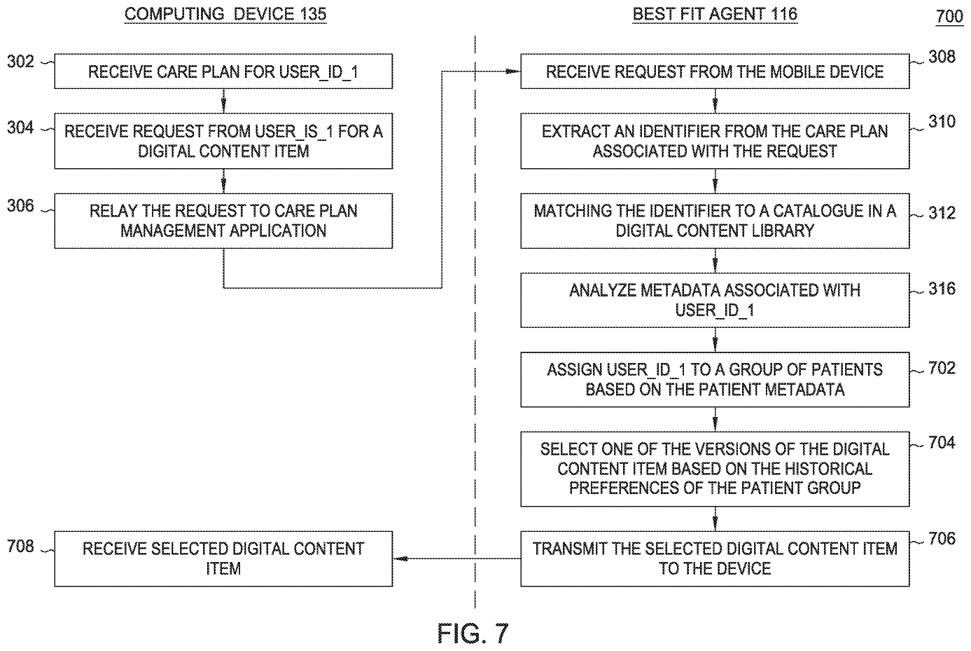

FIG. 7 illustrates a method 700 for delivering best fit content, according to one embodiment. The method 700 includes the blocks 302-314 from FIG. 3. After the best fit agent 116 analyzes the metadata associated with User_ID_1, the best fit agent 116 assigns User_ID_1 to a group of patients based on the patient metadata (block 702). For example, the best fit agent 116 may group User_ID_1 to a group of patients with similar metadata, such as education level, age, sex, etc. The best fit agent 116 then selects the version of content to be provided to the user based on historical preferences of the group (block 704). For example, the best fit agent 116 analyzes whether the group selects a particular form of content more often (e.g., a high sophistication content, video as opposed to text, etc.). The best fit agent 116 then pushes the content item to the mobile device (block 706). The mobile device receives the digital content item from the best fit agent 116 (block 708).

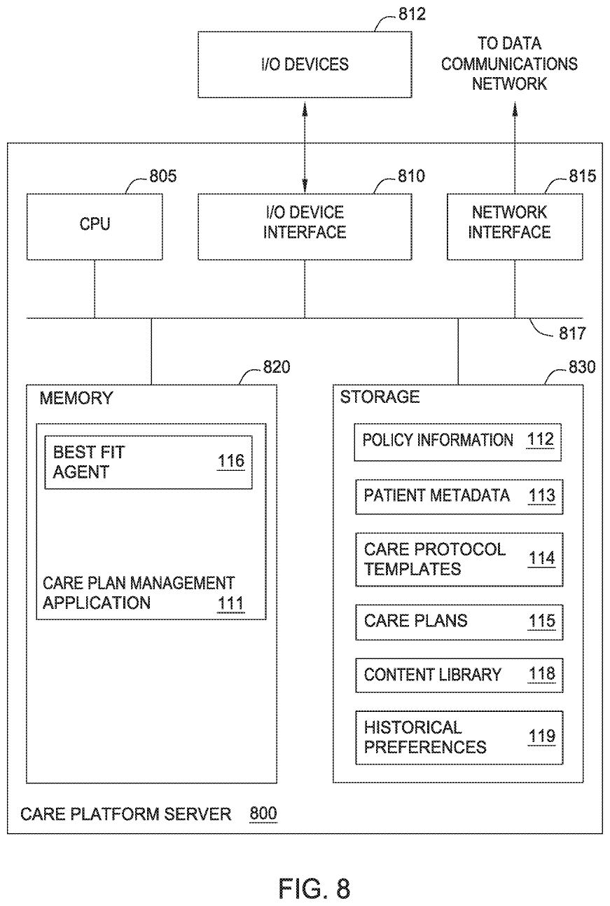

FIG. 8 illustrates a care platform server 800 configured to administer best fit content in the individualized care plan for a patient, according to one embodiment. As shown, the care platform server 800 includes, without limitation, a central processing unit (CPU) 805, a network interface 815, a memory 820, and storage 830, each connected to a bus 817. The care platform server may also include an I/O device interface 810 connecting I/O devices 812 (e.g., keyboard, display and mouse devices) to the care platform server 800. Further, in context of this disclosure, the computing elements shown in the care platform server 600 may correspond to a physical computing system (e.g., a system in a data center) or may be a virtual computing instance executing within a computing cloud.

CPU 805 retrieves and executes programming instructions stored in memory 820 as well as stores and retrieves application data residing in the storage 830. The bus 817 is used to transmit programming instructions and application data between CPU 805, I/O devices interface 810, storage 830, network interface 817, and memory 820. Note, CPU 805 is included to be representative of a single CPU, multiple CPUs, a single CPU having multiple processing cores, and the like. Memory 820 is generally included to be representative of a random access memory. Storage 830 may be a disk drive storage device. Although shown as a single unit, storage 830 may be a combination of fixed and/or removable storage devices, such as fixed disc drives, removable memory cards, or optical storage, network attached storage (NAS), or a storage area-network (SAN).

Illustratively, memory 820 includes a care plan management application 111. And storage 830 includes policy information 112, patient metadata 113, care protocol templates 114, care plans 115, and content library 118. The care plan management application 111 further includes the best fit agent 116. The best fit agent 116 is configured to determine which version of information in corresponding catalogue of the content library 118 is a “best fit information” for the user requesting a digital content item based on the patient metadata 113. The best fit agent 116 does this by looking at the patient metadata 113, and determining which version of information is most appropriate for the user. For example, the best fit agent 116 may determine that the version of “Generic Diabetes Information” that is an audio version may be the digital content item for a person with a hearing deficiency. In another example, the best fit agent 116 may determine that the version of “Generic Diabetes Information” that is detailed with scientific information may be the digital content item for a person with a post-doctorate education.

One embodiment of the present disclosure is implemented as a program product for use with a computer system. The program(s) of the program product defines functions of the embodiments (including the methods described herein) and can be contained on a variety of computer-readable storage media. Examples of computer-readable storage media include (i) non-writable storage media (e.g., read-only memory devices within a computer such as CD-ROM or DVD-ROM disks readable by an optical media drive) on which information is permanently stored; (ii) writable storage media (e.g., floppy disks within a diskette drive or hard-disk drive) on which alterable information is stored. Such computer-readable storage media, when carrying computer-readable instructions that direct the functions of the present disclosure, are embodiments of the present disclosure. Other examples media include communications media through which information is conveyed to a computer, such as through a computer or telephone network, including wireless communications networks.

In general, the routines executed to implement the embodiments of the present disclosure may be part of an operating system or a specific application, component, program, module, object, or sequence of instructions. The computer program of the present disclosure is comprised typically of a multitude of instructions that will be translated by the native computer into a machine-readable format and hence executable instructions. Also, programs are comprised of variables and data structures that either reside locally to the program or are found in memory or on storage devices. In addition, various programs described herein may be identified based upon the application for which they are implemented in a specific embodiment of the disclosure. However, it should be appreciated that any particular program nomenclature that follows is used merely for convenience, and thus the present disclosure should not be limited to use solely in any specific application identified and/or implied by such nomenclature.

As described, embodiments include receiving a request specifying an identifier for a digital content item to be delivered as part of administering an individualized care plan for a patient. The processor matches the document identifier to a catalogue in a digital content library. The catalogue includes a plurality of versions of the digital content item. The processor selects one of the versions of the digital content item in the catalogue based on metadata for the patient. The patient metadata includes personal information about the patient. The processor transmits the selected digital content item to a device of the patient.

While the foregoing is directed to embodiments of the present disclosure, other and further embodiments of the disclosure may be devised without departing from the basic scope thereof, and the scope thereof is determined by the claims that follow.

What is claimed is:

1. A method of administering information in a care plan to a patient, the method comprising:
receiving a request specifying an identifier for a digital content item to be delivered as part of administering an individualized care plan for a patient;
matching the document identifier to a catalogue in a digital content library, wherein the catalogue includes a plurality of versions of the digital content item;
selecting one of the versions of the digital content item in the catalogue based on patient metadata for the patient, wherein the patient metadata includes personal information about the patient; and
transmitting the selected digital content item to a device of the patient.

2. The method of claim 1, selecting one of the versions of the digital content item in the catalogue based on patient metadata for the patient, wherein each item of the patient metadata comprises a metadata parameter and a value associated with the metadata parameter, comprises:
assigning a weight to each value associated with each metadata parameter; and
choosing a best fit content item based on the weights.

3. The method of claim 2, further comprising:
determining whether a conflict exists between the selected digital content item and an item of patient metadata;
responsive to determining that the conflict exists, adjusting the weights assigned to each item of patient metadata; and
choosing another digital content item based on the updated weights assigned to each item of patient metadata.

4. The method of claim 2, wherein selecting one of the versions of the digital content item in the catalogue based on patient metadata for the patient comprises:
providing a point system to each value in the patient metadata; and
choosing the version in the catalogue that obtains a highest point total when the weights are assigned.

5. The method of claim 2, wherein selecting one of the versions of the digital content item in the catalogue based on patient metadata for the patient comprises:
choosing the version in the catalogue that corresponds to a highest ranking item of metadata.

6. The method of claim 1, further comprising:
receiving a second request specifying a second identifier for a second digital content item to be delivered as part of administering the individualized care plan for the patient;
matching the second document identifier to a second catalogue in the digital content library, wherein the second catalogue includes a plurality of versions of the second digital content item;
selecting one of the versions of the second digital content item in the second catalogue based on patient metadata for the patient;
localizing the second digital content item;
determining the specifications of the device to which the second digital content item will be provided;
optimizing the second digital content item based on the determined specifications of the device; and
transmitting the second digital content item to the device of the patient.

7. The method of claim 1, wherein the personal information defining the patient metadata includes age, sex, location, medical history, educational history, medical state, and institutional history.

8. The method of claim 1, further comprising:
receiving a second request specifying a second identifier for a second digital content item to be delivered as part of administering the individualized care plan for the patient;
matching the second document identifier to a second catalogue in the digital content library, wherein the second catalogue includes a plurality of versions of the second digital content item;
assigning the patient to a group of patients based on the patient metadata;
selecting the second digital content item for the patient based off historical information for that group of users; and
transmitting the second digital content item to the device of the patient.

9. The method of claim 1, further comprising:
receiving a second request specifying a second identifier for a second digital content item to be delivered as part of administering the individualized care plan for the patient;
matching the second document identifier to a second catalogue in the digital content library, wherein the second catalogue includes a plurality of versions of the second digital content item;
selecting the second digital content item for the patient based on historical preferences of the patient for other digital content items; and
transmitting the second digital content item to the device of the patient.

10. A system, comprising:
a computer processor; and
a memory containing a program that, when executed on the computer processor, performs an operation of administering information in a care plan to a patient, the operation comprising:
receiving a request specifying an identifier for a digital content item to be delivered as part of administering an individualized care plan for a patient;
matching the document identifier to a catalogue in a digital content library, wherein the catalogue includes a plurality of versions of the digital content item;
selecting one of the versions of the digital content item in the catalogue based on patient metadata for the patient, wherein the patient metadata includes personal information about the patient; and
transmitting the selected digital content item to a device of the patient.

11. The system of claim 10, wherein selecting one of the versions of the digital content item in the catalogue based on patient metadata for the patient, wherein each item of patient metadata comprises a metadata parameter and a value associated with the metadata parameter, comprises:
assigning a weight to each value associated with each metadata parameter; and
choosing a best fit content item based on the weights.

12. The system of claim 11, further comprising:
determining whether a conflict exists between the selected digital content item and an item of patient metadata;
responsive to determining that the conflict exists, adjusting the weights assigned to each item of patient metadata; and
choosing another digital content item based on the updated weights assigned to each item of patient metadata.

13. The system of claim 11, wherein selecting one of the versions of the digital content item in the catalogue based on patient metadata for the patient comprises:
providing a point system to each value in the patient metadata; and
choosing the version in the catalogue that obtains a highest point total when the weights are assigned.

14. The system of claim 11, wherein selecting one of the versions of the digital content item in the catalogue based on patient metadata for the patient comprises:
choosing the version in the catalogue that corresponds to a highest ranking item of metadata.

15. The system of claim 10, further comprising:
receiving a second request specifying a second identifier for a second digital content item to be delivered as part of administering the individualized care plan for the patient;
matching the second document identifier to a second catalogue in the digital content library, wherein the second catalogue includes a plurality of versions of the second digital content item;
selecting one of the versions of the second digital content item in the second catalogue based on patient metadata for the patient;
localizing the second digital content item;
determining the specifications of the device to which the second digital content item will be provided;
optimizing the second digital content item based on the determined specifications of the device; and
transmitting the second digital content item to the device of the patient.

16. The system of claim 10, wherein the personal information defining the patient metadata includes age, sex, location, medical history, educational history, medical state and institutional history.

17. The system of claim 10, further comprising:
receiving a second request specifying a second identifier for a second digital content item to be delivered as part of administering the individualized care plan for the patient;
matching the second document identifier to a second catalogue in the digital content library, wherein the second catalogue includes a plurality of versions of the second digital content item;
assigning the patient to a group of patients based on the patient metadata;
selecting the second digital content item for the patient based off historical information for that group of users; and
transmitting the second digital content item to the device of the patient.

18. The system of claim 10, further comprising:
receiving a second request specifying a second identifier for a second digital content item to be delivered as part of administering the individualized care plan for the patient;
matching the second document identifier to a second catalogue in the digital content library, wherein the second catalogue includes a plurality of versions of the second digital content item;
selecting the second digital content item for the patient based on historical preferences of the patient for other digital content items; and
transmitting the second digital content item to the device of the patient.

19. A computer readable medium for performing an operation of administering information in a care plan to a patient, comprising:
a computer readable storage medium having computer readable program code embodied therewith, the computer readable program code, comprising:
computer readable program code to receive a request specifying an identifier for a digital content item to be delivered as part of administering an individualized care plan for a patient;
computer readable program code to match the document identifier to a catalogue in a digital content library, wherein the catalogue includes a plurality of versions of the digital content item;
computer readable program code to select one of the versions of the digital content item in the catalogue based on patient metadata for the patient, wherein the patient metadata includes personal information about the patient; and
computer readable program code to transmit the selected digital content item to a device of the patient.

20. The computer readable medium of claim 19, wherein computer readable program code to select one of the versions of the digital content item in the catalogue based on patient metadata for the patient, wherein each item of patient metadata comprises a metadata parameter and a value associated with the metadata parameter, comprises:
assigning a weight to each value associated with each metadata parameter; and
choosing a best fit content item based on the weights.
